(19) United States
(12) Patent Application Publication
(10) Pub. No.: US 2024/0366411 A1
(43) Pub. Date: Nov. 7, 2024

(54) PAD FOR AN ACHILLES TENDON BANDAGE OR ACHILLES TENDON ORTHOSIS

(71) Applicant: BAUERFEIND AG, Zeulenroda-Triebes (DE)

(72) Inventors: Madlin Grundmann, Zeulenroda-Triebes (DE); Hans B. Bauerfeind, Zeulenroda-Triebes (DE)

(21) Appl. No.: 18/686,858

(22) PCT Filed: Aug. 12, 2022
(86) PCT No.: PCT/EP2022/072730

§ 371 Date: Feb. 26, 2024

(30) Foreign Application Priority Data
Aug. 27, 2021 (DE) 10 2021 209 453.8

(51) Int. Cl. A61F 5/01; A61F 5/30

(52) U.S. Cl. CPC A61F 5/0111; A61F 5/30

(57) ABSTRACT

The present invention relates to a pad for Achilles tendon bandages or orthoses, to an Achilles tendon bandage or orthosis which has a pad according to the invention, and to the use of a pad according to the invention for draining lymphatic fluid and for relieving the Achilles tendon.

### CROSS REFERENCE TO RELATED APPLICATIONS

This application is a U.S. a national stage application under 35 U.S.C. 371 of PCT/EP2022/072730 (“the ′730 PCT application”), filed Aug. 12, 2022, which claims priority to German patent application DE 10 2021 209 453.8 filed Aug. 27, 2021 (“the ′453.8 Priority Application”). The contents of the ′730 PCT Application and ′453.8 Priority Application are incorporated herein in their entireties.

### BACKGROUND

The present invention relates to a pad for Achilles tendon bandages or orthoses, to an Achilles tendon bandage or orthosis which has a pad according to the invention, and to the use of a pad according to the invention for draining lymphatic fluid and relieving the Achilles tendon.

The Achilles tendon is the thickest and strongest tendon in the human body. The tendon connects the heel bone (calcaneus) with the calf muscles and enables, among other things, a powerful flexion of the foot in the direction of the sole of the foot (planar flexion) as well as the upward tilting of the foot (supination). When running and in particular when jumping and landing, the Achilles tendon is exposed to very high forces. Acute or chronic irritation and inflammation of the Achilles tendon is a common problem in people who are active in sports.

Long-term incorrect strain and overstrain can lead to structural damage to the Achilles tendon. Painful damage to the Achilles tendon, known as Achillodynia, is typically characterised by recurring, strain-dependent pain in the area of the back of the lower leg and heel. Another painful condition of the Achilles tendon is inflammation of the bursa between the heel bone and Achilles tendon (Achillobursitis). The main symptoms of Achillobursitis are severe pain in the area of the Achilles tendon insertion. If the pain persists over a period of time, it can lead to a thickening of the heel bone, known as Haglund's exostosis (Haglund's heel).

Stabilisation and relief of the Achilles tendon is crucial for the prevention and targeted treatment of acute and chronic irritation and inflammation of the Achilles tendon as well as for post-traumatic and post-operative protection, for example after an Achilles tendon rupture. An Achilles tendon bandage produced from an elastic material with a cushion enclosing the Achilles tendon is described in DE 20 2004 013 159. A pad for an Achilles tendon bandage with a periodic, in particular wave-shaped, surface structure for reducing swelling and supporting the drainage of lymphatic fluid is known from DE 10 2008 015 449.

### BRIEF DESCRIPTION OF THE SEVERAL VIEWS OF THE DRAWINGS

FIG. 1A shows a schematic representation of the front surface of the pad according to the disclosure:

FIG. 1B schematically shows the rear surface of the pad of FIG. 1A;

FIG. 2 schematically shows the front surface of another embodiment of a pad according to the disclosure:

FIG. 3 shows an embodiment of the pad according to the disclosure in the applied state as viewed from the centre back of the ankle:

FIG. 4 schematically shows an embodiment of the pad according to the disclosure in the applied state when viewed from the rear at an angle to the ankle; and

FIG. 5 schematically shows an embodiment of the pad according to the disclosure in the applied state when viewed from the front at an angle to the ankle.

### DETAILED DESCRIPTION

The present invention is based on the task of providing an improved pad for use in Achilles tendon bandages or orthoses, with which stabilisation of the Achilles tendon can be achieved, the drainage of lymphatic fluid is improved and oedema formation is suppressed.

The present invention solves the underlying technical problem by the subject matter of the independent claims.

Herein, the invention relates to a pad for an Achilles tendon bandage or orthosis, wherein the pad comprises a front surface, a rear surface and two lower pad arms, and wherein each of the lower pad arms has at least one pressure element on the front surface.

In a particularly preferred embodiment of the present invention, the lower pad arms are formed to run below the malleoli in the applied state of the pad. According to a preferred embodiment, the lower pad arms are formed to run on guide tracks below the malleoli in the applied state of the pad.

Preferably, in the applied state of the pad, the at least one pressure element arranged on each of the two lower pad arms exerts pressure on the tissue below the malleoli, in particular on Bisgaard regions. Preferably, the at least one pressure element arranged on each of the two lower pad arms lies in the applied state of the pad on pathways below the malleoli.

Gripping around the malleoli and compressing the tissue below the malleoli, in particular Bisgaard regions, stabilises the ankle, in particular the Achilles tendon and the heel bone, improves the removal of lymphatic fluid and reduces or prevents the formation of oedema.

In a particularly preferred embodiment of the present invention, each of the lower pad arms has exactly one pressure element on the front surface. According to this preferred embodiment of the invention, the pad comprises a total of two pressure elements. According to a preferred embodiment of the present invention, each of the lower pad arms thus has a single pressure element on the front surface.

Preferably, the at least one pressure element is a protrusion. Particularly preferably, the at least one pressure element is a cusps-like protrusion. In a preferred embodiment, the at least one pressure element is not a nub, in particular not a nub as disclosed below as additional nubs. In an alternative embodiment, the at least one pressure element is a nub. According to a further preferred embodiment of the present invention, the at least one pressure element is a plateau-like protrusion with rounded edges.

In a particularly preferred embodiment of the present invention, it may be provided that the at least one pressure element has a nubbing on its surface. Preferably, the at least one pressure element has at least two, preferably at least three, preferably at least four, preferably at least five, preferably at least six nubs on its surface.

According to a preferred embodiment, the at least one pressure element has an elliptical or round cross-section.

Preferably, the at least one pressure element has a height of at least 2 mm, preferably at least 3 mm, preferably at least 4 mm, preferably at least 5 mm, preferably at least 6 mm, preferably at least 7 mm, preferably at least 8 mm, preferably at least 9 mm, preferably at least 10 mm, starting from its origin. Particularly preferably, the at least one pressure element has a height of 2 to 10 mm, preferably 3 to 9) mm, preferably 4 to 8 mm, starting from its origin. Preferably, the at least one pressure element has a height of at most 10 mm, preferably at most 9 mm, preferably at most 8 mm, preferably at most 7 mm, preferably at most 6 mm, preferably at most 5 mm, starting from its origin.

In a further preferred embodiment of the present invention, the at least one pressure element has a diameter at its origin of at least 7 mm, preferably at least 8 mm, preferably at least 9 mm, preferably at least 10 mm, preferably at least 12 mm, preferably at least 14 mm, preferably at least 16 mm, preferably at least 18 mm, preferably at least 20 mm, preferably at least 22 mm, preferably at least 24 mm, preferably at least 26 mm, preferably at least 28 mm, preferably at least 30 mm. Preferably, the at least one pressure element has a diameter at its origin of 10 to 40 mm, preferably 12 to 36 mm, preferably 14 to 32 mm, preferably 16 to 28 mm, preferably 18 to 24 mm. In a further preferred embodiment, the at least one pressure element has a diameter at its origin of at most 40 mm, preferably at most 38 mm, preferably at most 36 mm, preferably at most 34 mm, preferably at most 32 mm, preferably at most 30 mm, preferably at most 28 mm, preferably at most 26 mm, preferably at most 24 mm, preferably at most 22 mm, preferably at most 20 mm.

According to a preferred embodiment of the present invention, the at least one pressure element has a uniform cross-section, in particular a uniform diameter, starting from its origin to its end. According to a further embodiment, the at least one pressure element does not have a uniform cross-section, in particular a uniform diameter, starting from its origin to its end. Preferably, the cross-section, in particular the diameter, of the at least one pressure element changes starting from its origin to its end. In a preferred embodiment of the present invention, the cross-section, in particular the diameter, of the at least one pressure element changes continuously starting from its origin to its end. Preferably, the cross-section, in particular the diameter, of the at least one pressure element changes discontinuously starting from its origin to its end. Preferably, the at least one pressure element has a larger diameter at its origin than at its end.

According to a preferred embodiment of the present invention, each of the lower pad arms has a single pressure element on the front surface, wherein the lower pad arms run below the malleoli in the applied state of the pad and pressure is exerted on the tissue below the malleoli by the one pressure element arranged on each of the two lower pad arms, wherein the one pressure element is a plateau-like protrusion with rounded edges.

According to a preferred embodiment of the present invention, the front surface of the pad is nubbed.

In a preferred embodiment, the nubs on the front surface of the pad have an elliptical or round cross-section.

In a preferred embodiment, the nubs on the front surface of the pad have a cylindrical shape. Preferably, the nubs on the front surface of the pad have a conical shape. Particularly preferably, the nubs on the front surface of the pad have a rounded, cusps-like shape.

Particularly preferably, the front surface of the pad comprises at least four, preferably at least six, preferably at least 10, preferably at least 12, preferably at least 14, preferably at least 16, preferably at least 18, preferably at least 20, preferably at least 30, preferably at least 40, preferably at least 50, nubs.

In a preferred embodiment of the present invention, the nubs on the front surface of the pad have a height of at least 0.5 mm, preferably at least 1 mm, preferably at least 1.5 mm, preferably at least 2 mm, preferably at least 2.5 mm, preferably at least 3 mm, preferably at least 3.5 mm, preferably at least 4 mm, starting from their origin. Particularly preferably, the nubs on the front surface of the pad have a height of 0.5 to 10 mm, preferably 0.5 to 7.5 mm, preferably 0.5 to 5 mm, preferably 0.5 to 4 mm, preferably 1 to 4 mm, preferably 0.5 to 3.5 mm, preferably 1 to 3.5 mm, preferably 0.5 to 3 mm, preferably 1 to 3 mm, starting from their origin. Preferably, the nubs on the front surface of the pad have a height of at most 10 mm, preferably at most 9 mm, preferably at most 8 mm, preferably at most 7 mm, preferably at most 6 mm, preferably at most 5 mm, preferably at most 4.5 mm, preferably at most 4 mm, preferably at most 3.5 mm, preferably at most 3 mm, starting from their origin.

According to a further preferred embodiment of the present invention, the nubs on the front surface of the pad have a diameter at their origin of at least 1 mm, preferably at least 1.5 mm, preferably at least 2 mm, preferably at least 2.5 mm, preferably at least 3 mm, preferably at least 3.5 mm, preferably at least 4 mm, preferably at least 4.5 mm, preferably at least 5 mm. Preferably, the nubs on the front surface of the pad have a diameter at their origin of 1 to 8 mm, preferably 1 to 7 mm, preferably 1 to 6 mm, preferably 2 to 5 mm, preferably 3 to 5 mm. In another preferred embodiment, the nubs on the front surface of the pad have a diameter at their origin of at most 8 mm, preferably at most 7.5 mm, preferably at most 7 mm, preferably at most 6.5 mm, preferably at most 6 mm, preferably at most 5.5 mm, preferably at most 5 mm, preferably at most 4.5 mm, preferably at most 4 mm, preferably at most 3.5 mm, preferably at most 3 mm.

In a particularly preferred embodiment of the present invention, the nubs on the front surface of the pad have a uniform cross-section, in particular a uniform diameter, starting from their origin to their end. According to a further embodiment, the nubs on the front surface of the pad do not have a uniform cross-section, in particular a uniform diameter starting from their origin to their end. Thus, it may be provided that the cross-section, in particular the diameter, of the nubs on the front surface of the pad changes from its origin to its end. Preferably, the nubs on the front surface of the pad have a larger diameter at their origin than at their end.

In a further preferred embodiment of the present invention, the individual nubs on the front surface of the pad have a distance of at least 1 mm, preferably at least 1.25 mm, preferably at least 1.5 mm, preferably at least 1.75 mm, preferably at least 2 mm, from each other. Preferably, the individual nubs on the front surface of the pad have a distance of at most 15 mm, preferably at most 10 mm, preferably at most 9 mm, preferably 8 mm, preferably at most 7 mm, preferably at most 6 mm, preferably at most 5 mm, from one another. Preferably, the distance of a nub on the front surface of the pad from the nearest nub is not less than half the height of the nearest nub starting from their origin. Particularly preferably, the distance of a nub on the front surface of the pad from the nearest nub is not less than half the diameter of the nearest nub at their origin.

Preferably, the nubs on the front surface of the pad extend over the lower pad arms. Particularly preferably, at least one nub is arranged distally from the at least one pressure element on each of the lower pad arms. Preferably, at least two nubs, preferably at least three nubs, preferably at least four nubs, are arranged distally from the at least one pressure element on each of the lower pad arms. The massage effect exerted on Bisgaard regions in the applied state of the pad can be further enhanced by the nubs preferably arranged on each of the lower pad arms distal from the at least one pressure element.

In a further preferred embodiment, the nubs on the front surface of the pad extend over the upper pad arms. Particularly preferably, the nubs on the front surface of the pad extend over the lower and upper pad arms.

According to a preferred embodiment of the present invention, the nubs on the front surface of the pad are arranged mirror symmetrical with respect to a mirror plane running vertically in the central area of the pad.

The nubs on the front surface of the pad cause an alternating pressure massage of the underlying tissue when worn. The massage effect advantageously leads to, among other things, the flushing out of any oedema that may be present and promotes local blood circulation, whereby both a pain-relieving and healing-promoting effect is achieved. In addition, the nubs arranged on the front surface of the pad, particularly in combination with a perforation in the pad, ensure that sufficient air circulation between the pad and the wearer's skin is guaranteed in the applied state of the pad.

Preferably, therefore, the nubs of the nubbing are not pressure elements, in particular not pressure elements in the sense of the invention, but rather the nubs of the nubbing are preferably massage elements.

Preferably, the nubs are smaller than the pressure elements. Preferably, the nubs have a smaller diameter than the pressure elements. Preferably, the nubs have a smaller height than the pressure elements. Preferably, the nubs have a smaller diameter and a smaller height than the pressure elements.

According to a further preferred embodiment of the present invention, the pad has a perforation.

In a particularly preferred embodiment, the perforation of the pad has at least two, preferably at least four, preferably at least six, preferably at least eight, preferably at least 10, preferably at least 12, preferably at least 14, preferably at least 16, preferably at least 18, preferably at least 20, preferably at least 30, preferably at least 40 holes.

In a further preferred embodiment, the individual holes of the perforation are arranged adjacent to the nubs present on the front surface of the pad. Preferably, the individual holes of the perforation are located at a distance of at most 10 mm, preferably at most 8 mm, preferably at most 6 mm, preferably at most 5 mm, preferably at most 4 mm, preferably at most 3 mm, preferably at most 2 mm, preferably at most 1 mm, from an adjacent nub arranged on the front surface of the pad, in particular from the nearest nub arranged on the front surface of the pad.

According to a preferred embodiment of the present invention, the holes of the perforation have a diameter of at least 1 mm, preferably at least 1.5 mm, preferably at least 2 mm, preferably at least 2.5 mm, preferably at least 3 mm, preferably at least 3.5 mm, preferably at least 4 mm, preferably at least 4.5 mm, preferably at least 5 mm. In a further preferred embodiment of the invention, the holes of the perforation have a diameter of 1 to 10 mm, preferably 2 to 8 mm, preferably 3 to 7 mm, preferably 4 to 6 mm. Particularly preferably, the holes of the perforation have a diameter of at most 10 mm, preferably at most 9 mm, preferably at most 8 mm, preferably at most 7 mm, preferably at most 6 mm, preferably at most 5 mm, preferably at most 4 mm, preferably at most 3 mm, preferably at most 2 mm.

The perforation of the pad, in particular in combination with a nubbing of the front surface of the pad, advantageously ensures ventilation of the skin covered by the pad in the applied state. This prevents or at least reduces perspiration along the Achilles tendon and in the ankle joint area while the pad, in particular an Achilles tendon bandage or orthosis comprising the pad, is being worn.

According to a preferred embodiment of the invention, the lower pad arms have a perforation. Preferably, the upper pad arms have a perforation. Particularly preferably, the upper and lower pad arms have a perforation.

In a further preferred embodiment of the invention, the lower pad arms have no perforations. Preferably, the upper pad arms have no perforations. Particularly preferably, the upper and lower pad arms have no perforations.

In a particularly preferred embodiment of the present invention, the pad is mirror symmetrical.

According to a further preferred embodiment, the pad has a material taper. Preferably, the material taper runs in a vertical direction in the applied state of the pad. Particularly preferably, the material taper runs in the area of a vertically running mirror plane of the pad.

In a particularly preferred embodiment, the material taper is characterised by the fact that the material thickness of the pad in the area of the material taper is at most 5 mm, preferably at most 4.5 mm, preferably at most 4 mm, preferably at most 3.5 mm, preferably at most 3 mm, preferably at most 2.5 mm, preferably at most 2 mm, preferably at most 1.5 mm, preferably at most 1 mm. Preferably, the pad has a material thickness in the area of the material taper of 1 to 5 mm, preferably 1 to 4.5 mm, preferably 1 to 4 mm, preferably 1 to 3.5 mm, preferably 1 to 3 mm, preferably 1 to 2.5 mm, preferably 1.5 to 2 mm, preferably 1.5 to 2 mm. In a further preferred embodiment of the present invention, the material thickness of the pad in the area of the material taper is at least 0.5 mm, preferably at least 0.75 mm, preferably at least 1 mm, preferably at least 1.25 mm, preferably at least 1.5 mm, preferably at least 1.75 mm, preferably at least 2 mm, preferably at least 2.25 mm, preferably at least 2.5 mm.

In the applied state of the pad, the area of the material taper clings itself to the Achilles tendon and grips around it. This advantageously results in additional stabilisation and relief of the Achilles tendon.

In a particularly preferred embodiment of the invention, the pad is formed from at least one elastic material, in particular from at least one viscoelastic material. Different materials suitable for producing an elastic, in particular viscoelastic, pad are known to the person skilled in the art.

In a preferred embodiment, the at least one elastic material, in particular the at least one viscoelastic material, is silicone. In a further preferred embodiment, the at least one elastic material, in particular the at least one viscoelastic material, is rubber, in particular natural or synthetic rubber. Further examples of elastic materials which are suitable for use in a pad according to the invention are thermoplastic elastomers, neoprene, synthetic latex or natural latex. According to the invention, it may also be provided that the pad comprises a mixture of different materials, in particular a mixture of at least two materials.

According to a preferred embodiment of the present invention, the pad has at least one upper pad arm in addition to the two lower pad arms. Preferably, the pad has two lower pad arms and two upper pad arms. Particularly preferably, the two lower pad arms are longer than the at least one upper pad arm, in particular longer than the two upper pad arms.

According to a particularly preferred embodiment of the present invention, the at least one upper pad arm, in particular the two upper pad arms, extends to the base of the calf muscles in the applied state of the pad.

The present invention also relates to an Achilles tendon bandage or orthosis comprising a pad according to the invention.

Preferably, the Achilles tendon bandage or orthosis is an elastic knitted bandage or elastic knitted orthosis.

In a preferred embodiment of the present invention, the pad according to the invention is an integral part of the Achilles tendon bandage or orthosis. According to this embodiment, the pad according to the invention cannot be separated from the Achilles tendon bandage or orthosis in a non-destructive manner. In particular, it may be provided that the pad according to the invention is sewn into the textile material of an Achilles tendon bandage or orthosis.

According to a further preferred embodiment of the present invention, the pad according to the invention is not an integral part of the Achilles tendon bandage or orthosis. According to this preferred embodiment, the pad according to the invention can be removed from the Achilles tendon bandage or orthosis in a non-destructive manner. In particular, it may be provided that the pad according to the invention can be inserted into a receptacle provided for this purpose, in particular a pocket, of the textile material of an Achilles tendon bandage or orthosis and can be removed from the latter again.

The present invention also relates to a use of a pad according to the invention for draining lymphatic fluid and relieving the Achilles tendon, in particular the non-therapeutic use of a pad according to the invention for draining lymphatic fluid and relieving the Achilles tendon.

The present invention also relates to the pad according to the invention for a use in a method for preventing and/or treating a functional disorder or injury of the Achilles tendon.

In a preferred embodiment of the present invention, the functional disorder or injury of the Achilles tendon is an acute or chronic irritation or inflammation of the Achilles tendon. Preferably, the functional disorder or injury of the Achilles tendon is selected from the group consisting of Achillobursitis, Achillodynia, Achilles tendon rupture and partial Achilles tendon rupture.

Further preferred embodiments of the present invention are the subject-matter of the sub-claims.

In the context of the present invention, the terms “comprising” and “having” are understood to mean that, in addition to the elements explicitly included by these terms, further elements not explicitly mentioned may be added. In the context of the present invention, these terms are also understood to mean that only the explicitly mentioned elements are included and that no further elements are present. In this particular embodiment, the meaning of the terms “comprising” and “having” is synonymous with the term “consisting of”. Furthermore, the terms “comprising” and “having” also include compositions which, in addition to the explicitly mentioned elements, also contain further elements which are not mentioned but which are of a functionally and qualitatively subordinate nature. In this embodiment, the terms “comprising” and “having” are synonymous with the term “consisting essentially of”.

In the context of the present invention, the term “and/or” is understood to mean that all members of a group which are connected by the term “and/or” are disclosed both alternatively to each other and cumulatively to each other in any combination. For the expression “A, B and/or C”, this means that the following disclosure content is to be understood: a) A or B or C or b) (A and B) or c) (A and C) or d) (B and C) or e) (A and B and C).

In the context of the present invention, the term “pathways” refers to the channels carrying vital energy running in the human body according to traditional Chinese medicine (TCM).

A “pad arm” refers to an extension extending from a base body of the pad into the periphery. The terms “lower pad arm”, “lower pad arms”, “upper pad arm” and “upper pad arms” are used in the context of the present invention to designate those extensions of the pad which, in the applied state of the pad, extend in a distal, in particular plantar, direction (“lower pad arms”) or extend in a proximal direction (“upper pad arms”), i.e, in the direction of the centre of the wearer's body, in particular running below (“lower pad arms”) or above (“upper pad arms”) the malleoli.

In the context of the present invention, the term “nubbing” refers to a plurality, in particular a variety, of regularly or irregularly arranged protrusions, in particular nubs.

In the context of the present invention, the term “perforation” refers to a plurality, in particular a variety, of regularly or irregularly arranged, locally limited material recesses extending from the front surface to the rear surface of the pad. Each individual hole of the “perforation” can have a uniform or non-uniform cross-section when viewed over the entire area of the material recess. For example, the material recess can have the geometric shape of a cylinder or truncated cone.

The term “Bisgaard region” refers to the region between the Achilles tendon, heel bone and ankle (Regio calcaneo-malleolaris) named according to the Danish physician Holger Bisgaard.

The present invention is illustrated below by means of exemplary figures. The inventive concept is not limited to these embodiments.

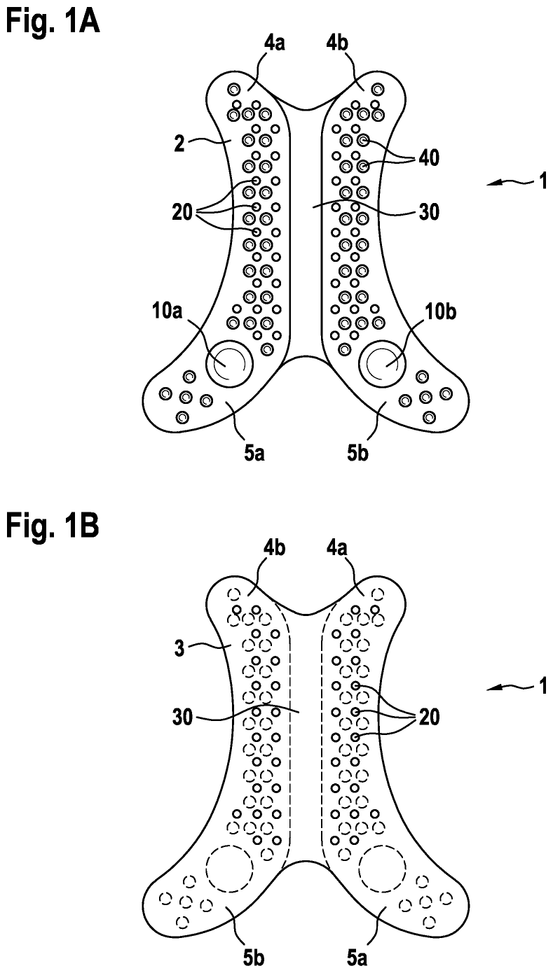

FIG. 1A shows a schematic representation of the front surface (2) of a preferred embodiment of the pad (1) according to the invention. The pad (1) has two upper pad arms (4a. 4b) and two lower pad arms (5a. 5b), wherein a pressure element (10a. 10b) is arranged on each of the two lower pad arms (5a, 5b), via which in the applied state the pad (1) pressure is exerted on Bisgaard regions when worn. In addition, the pad (1) comprises nubs (40) arranged on its front surface (2) and a perforation (20). In the area of its mirror plane, the pad (1) has a material taper (30) running in a vertical direction.

FIG. 1B schematically shows the rear surface (3) of the preferred embodiment of the pad (1) according to the invention shown in FIG. 1A.

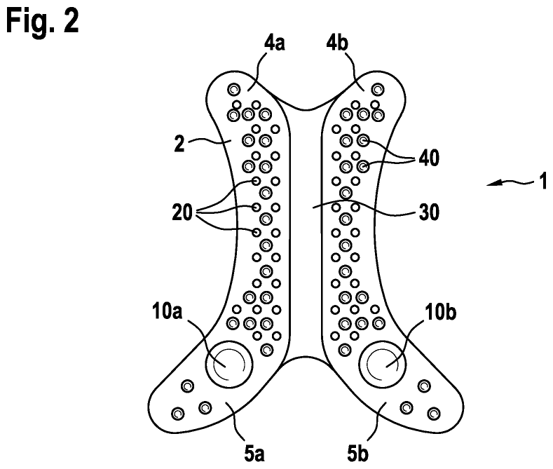

FIG. 2 schematically shows the front surface (2) of a further preferred embodiment of a pad (1) according to the invention. The pad (1) also has two upper pad arms (4a, 4b) and two lower pad arms (5a, 5b), wherein a pressure element (10a. 10b) is arranged on each of the two lower pad arms (5a, 5b), via which in the applied state of the pad (1) pressure is exerted on the Bisgaard regions when worn. Like the pad shown in FIG. 1A, the pad (1) shown in FIG. 2 also comprises nubs (40) arranged on its front surface (2) and a perforation (20), wherein the embodiment of a pad (1) according to the invention shown in FIG. 2 is narrower and has a smaller number of nubs (40) than the embodiment shown in FIG. 1A. The pad (1) is formed mirror symmetrical and has a material taper (30) running in a vertical direction in the area of the mirror plane.

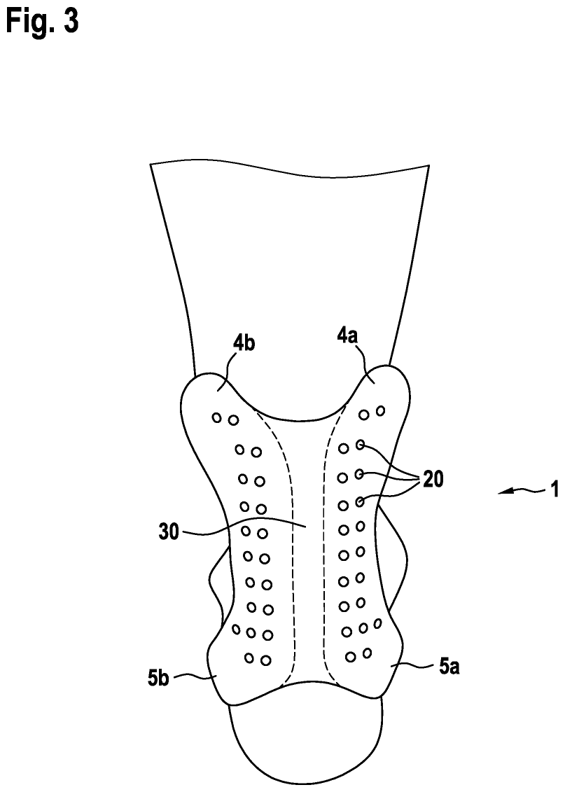

FIG. 3 shows a preferred embodiment of the pad (1) according to the invention in the applied state as viewed from the centre back of the ankle. It can be seen that the lower pad arms (5a, 5b) run below the malleoli. The upper pad arms (4a, 4b) of the presented embodiment extend in a proximal direction up to the insertion of the calf muscles. The perforation (20) in the pad (1) ensure sufficient air circulation when worn. In the central area of the pad (1), a material taper (30)) runs in a vertical direction, which is adapted to the surface contour of the Achilles tendon and grips it in a stabilising manner.

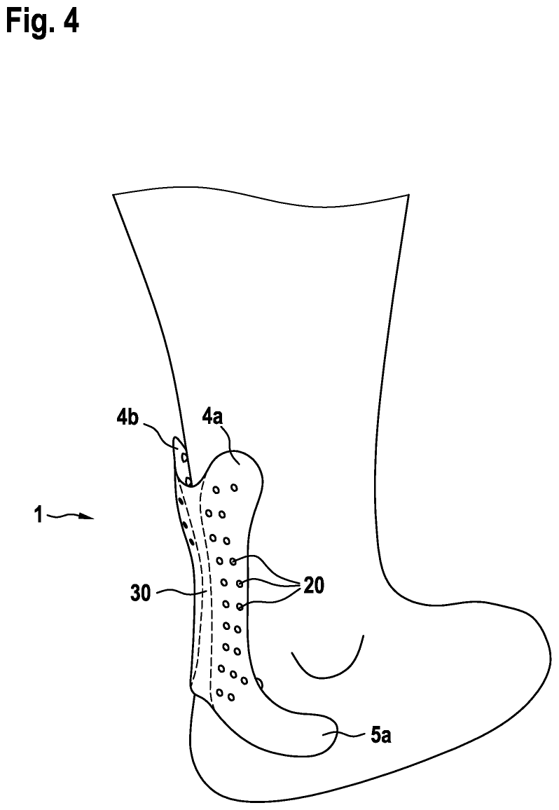

FIG. 4 schematically shows a preferred embodiment of the pad (1) according to the invention in the applied state when viewed from the rear at an angle to the ankle.

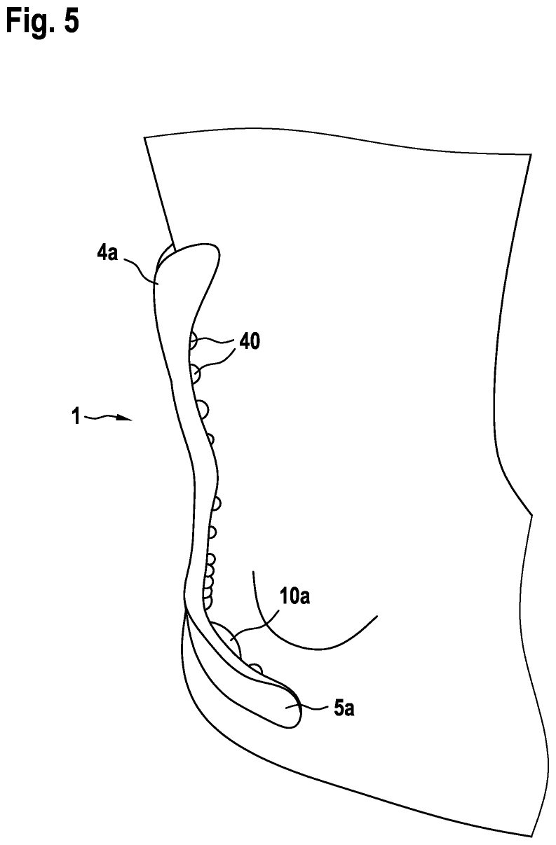

FIG. 5 schematically shows a preferred embodiment of the pad (1) according to the invention in the applied state when viewed from the front at an angle to the ankle. The nubs (40) of the pad (1) arranged on the front surface (2) provide a massage effect when worn and allow air circulation between the pad (1) and the underlying tissue. The lower pad arm (5a) runs below the external ankle (Malleolus lateralis) and has a pressure element (10a) which exerts pressure on the tissue in the Regio retromalleolaris lateralis between the external ankle (Malleolus lateralis) and the heel bone (calcaneus) when the pad (1) is worn. Also presented is an upper pad arm (4a) that extends to the insertion of the calf muscles.

### REFERENCE SIGNS

1 Pad for an Achilles tendon bandage or orthosis
2 Front surface of the pad
3 Rear surface of the pad
4a First upper pad arm
4b Second upper pad arm
5a First lower pad arm
5b Second lower pad arm
10a Pressure element
10b Pressure element
20) Perforation
30) Material taper
40) Nubs

1. A pad for an Achilles tendon bandage or orthosis, wherein the pad comprises a front surface, a rear surface and two lower pad arms, wherein each of the lower pad arms has at least one pressure element on the front surface which is a plateau-like protrusion with rounded edges, characterised in that that the lower pad arms fax run below the malleoli in an applied state of the pad and pressure is exerted on the tissue below the malleoli by the at least one pressure element arranged on each of the two lower pad arms.

2. The pad according to claim 1, characterised in that each of the lower pad arms has a single pressure element on the front surface.

3. The pad according to claim 1, characterised in that each of the lower pad arms has a single pressure element on the front surface, wherein the lower pad arms run below the malleoli in the applied state of the pad and pressure is exerted on the tissue below the malleoli by the one pressure element arranged on each of the two lower pad arms, wherein the one pressure element is a plateau-like protrusion with rounded edges.

4. The pad according to claim 1, characterised in that the front surface of the pad is nubbed.

5. The pad according to claim 1, characterised in that the pad has a perforation.

6. The pad according to claim 1, characterised in that the pad is mirror symmetrical.

7. The pad according to claim 6, characterised in that the pad has a material taper in an area of a mirror plane.

8. The pad according to claim 1, characterised in that the pad is formed from at least one elastic material.

9. The pad according to claim 1, characterised in that the pad has two upper pad arms and two lower pad arms.

10. The pad according to claim 9, wherein the lower pad arms (5a, 5b) are longer than the upper pad arms.

11. An Achilles tendon bandage or orthosis comprising a pad according to claim 1.

12. A use of a pad according to claim 1 for draining lymphatic fluid and relieving the Achilles tendon.

13. A pad for an Achilles tendon bandage comprising:
a front surface, a rear surface and two lower pad arms, wherein each of the two lower pad arms comprises a pressure element on the front surface which is a plateau-like protrusion with rounded edges,
wherein, when the pad is in an applied state, the two lower pad arms run below a malleoli of a user and exert pressure on the tissue below the malleoli by the pressure element on each of the two lower pad arms.

14. A method of draining lymphatic fluid and relieving the Achilles tendon, the method comprising:
selecting a pad comprising a front surface, a rear surface and two lower pad arms, wherein each of the two lower pad arms comprises a pressure element on the front surface which is a plateau-like protrusion with rounded edges;
applying the pad to a user such that the two lower pad arms run below a malleoli of the user and the pressure element on each of the two lower pad arms exert pressure on the tissue below the malleoli.
